(19) United States
(12) Patent Application Publication
(10) Pub. No.: US 2010/0030128 A1
(43) Pub. Date: Feb. 4, 2010

(54) IONTOPHORESIS DEVICE

(76) Inventors: Kazuma Mitsuguchi, Tokyo (JP); Mitsugu Yamauchi, Tokyo (JP); Mizuo Nakayama, Tokyo (JP); Takehiko Matsumura, Tokyo (JP); Hidero Akiyama, Tokyo (JP); Akihiko Matsumura, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/065,851

(22) PCT Filed: Sep. 1, 2006
(86) PCT No.: PCT/JP2006/317321

§ 371 Date: Apr. 17, 2009

(30) Foreign Application Priority Data
Sep. 6, 2005 (JP) 2005-258304

(51) Int. Cl. A61N 1/30

(52) U.S. Cl. 604/20

(57) ABSTRACT

The transport efficiency of a drug using an iontophoresis device is increased using a negative pressure device.

The iontophoresis device includes a working electrode assembly and a non-working electrode assembly for administering drug by iontophoresis. Each of the working electrode assembly and the non-working electrode assembly includes an adhesive surface configured to adhere to a biological interface. A DC electric power source is connected to the working electrode assembly and the non-working electrode assembly. The iontophoresis device further includes a negative pressure device integral with at least one of the working electrode assembly and the non-working electrode assembly and configured to maintain a negative pressure between the at least one of the working electrode assembly and the non-working electrode assembly and the biological interface.

### CROSS-REFERENCE TO RELATED APPLICATIONS

This application is the U.S. national stage of international application no. PCT/JP2006/317321, filed Sep. 1, 2006, which claims benefit from Japanese application no. 2005-258304, filed Sep. 6, 2005, which applications are hereby incorporated by reference in their entirety.

### BACKGROUND

1. Technical Field

The present description relates to an iontophoresis device including: a working electrode assembly and a non-working electrode assembly for administering a drug by iontophoresis; and a DC electric power source connected to the working electrode assembly and the non-working electrode assembly.

2. Description of the Related Art

Iontophoresis devices, such as those shown in JP 04-297277A, are well known. An iontophoresis device typically has an ion exchange membrane arranged to realize the efficient introduction of a drug (e.g., a drug ion) held by the device through a biological interface, such as a portion of skin or a mucous membrane.

However, an ion exchange membrane itself typically has no adhesiveness with biological interfaces. Accordingly, structures, such as those shown in FIGS. 5-7, have been implemented to mount iontophoresis devices.

FIG. 5 shows a conventional iontophoresis device 1, and FIG. 6 shows a surface of an electrode assembly 2 (such as the working electrode assembly 2a, or non-working electrode assembly 2b of FIG. 5).

The iontophoresis device 1 shown in each of FIGS. 5 and 6 will be briefly described.

The iontophoresis device 1 may comprise a working electrode assembly 2a and a non-working electrode assembly 2b connected to an electric power source 3. The working electrode assembly 2a includes an electrode 5a connected to the electric power source 3, a buffer solution holding portion 6a adjacent a front surface of the electrode 5a, an anion exchange membrane 7a adjacent a front surface of the buffer solution holding portion 6a, a drug holding portion 8a adjacent a front surface of the anion exchange membrane 7a, and a cation exchange membrane 9a adjacent a front surface of the drug holding portion 8a, all housed in a backing layer 4a. In this embodiment, the backing layer 4a doubles as a case.

In addition, an adhesive portion 10a may be arranged around the cation exchange membrane 9a to form part of an adhesive surface 11a of the working electrode assembly 2a, for causing the working electrode assembly 2a to adhere to a biological interface. A hydrogel or similar material is often used for the adhesive portion 10a. However, any material may be used to provide adhesion to the biological interface.

The non-working electrode assembly 2b includes an electrode 5a connected to the electric power source 3, a buffer solution holding portion 6b adjacent a front surface of the electrode 5b, a cation exchange membrane 7b adjacent a front surface of the buffer solution holding portion 6a, an electrolyte solution holding portion 8b adjacent a front surface of the cation exchange membrane 7b, and an anion exchange membrane 9b adjacent a front surface of the electrolyte solution holding portion 8b, all housed in a backing layer 4b. The backing layer 4b may also double as a case for the non-working electrode assembly 2b.

In addition, an adhesive portion 10b may be arranged around the anion exchange membrane 9b and on the side of an adhesive surface 11b of the non-working electrode assembly 2b.

In the case where one of the adhesive portions 10a or 10b is arranged on the side of a respective adhesive surface 11a or 11b of an electrode assembly 2a or 2b, the corresponding ion exchange membrane, which itself may have little or no adhesiveness, may be mounted to a biological interface (e.g., skin) while being in close contact with the biological interface even when the corresponding ion exchange membrane is placed on the side of the adhesive surface 11a or 11b.

For convenience of description, in each of FIGS. 5 and 6, the thickness and other dimensions of the iontophoresis device 1 have been exaggerated. Many such devices may have, for example, a structure with increased flatness, as shown in FIG. 7. lontophoresis devices have also been mounted to the skin by means of bands, tape, or external means, in the absence of an adhesive portion.

However, when one tries to mount a working or non-working electrode assembly using bands, the mounting may require a long time, and may be difficult. In addition, when the band is tightened incorrectly, the monted electrode assembly may be misaligned. When an electrode assembly is fixed to a biological interface using tape, a rash or other irritation of the biological interface due to the tape's adhesive may result.

On the other hand, where an adhesive portion is present on the adhesive surface of an electrode assembly as described above with reference to FIGS. 5 and 6, the above problems associated with bands or tape may not occur, but an adhesive strength of the assembly may be reduced after repeated application. In addition, an original adhesive strength may be insufficient in some cases. Even if a device is to be used only once (a so-called disposable device), although the reduction in adhesive strength due to repeated application is no longer a concern, there remains the problem of insufficient adhesive strength.

Meanwhile, a biological interface often has fine irregularities. When an electrode assembly is stuck to the biological interface, only a portion of the biological interface contacts the adhesive surface of the electrode assembly owing to the irregularities. It has been found that increasing the degree to which an iontophoresis device (more accurately, the adhesive surface/surfaces of a working electrode assembly and/or a non-working electrode assembly) and the biological interface adhere to each other enlarges the area where they are in contact with each other, and increases the amount of drug transported.

The presently disclosed embodiments may help to address the above problems and may improve the adhesion between a working electrode assembly and/or a non-working electrode assembly of an iontophoresis device and a biological interface.

The term “front surface” as used in the specification including the foregoing description, refers to the surface that is closer to a biological interface during use (e.g., mounting) of a device.

### BRIEF SUMMARY

In one embodiment, an iontophoresis device includes a working electrode assembly and a non-working electrode assembly for administering a drug by iontophoresis and a DC electric power source connected to the working electrode assembly and the non-working electrode assembly with polarities opposite to each other. Each of the working electrode assembly and the non-working electrode assembly may include an adhesive surface configured to adhere to a biological interface. The iontophoresis device may further include a negative pressure device integral with at least one of the working electrode assembly and the non-working electrode assembly and configured to maintain a negative pressure between the at least one of the working electrode assembly and the non-working electrode assembly and the biological interface.

This negative pressure device may increase the degree of adhesion between the adhesive surface/surfaces of the working electrode assembly and/or the non-working electrode assembly and the biological interface.

In one embodiment, the negative pressure device may include a suction cup at least partially surrounding the adhesive surface of the at least one of the working electrode assembly and the non-working electrode assembly. The suction cup may be configured to generate and maintain the negative pressure when the suction cup returns to an original shape due to an elastic restoring force of the suction cup after the suction cup is pressed in toward the biological interface.

Thus, the degree of adhesion between the adhesive surface/surfaces of the working electrode assembly and/or the non-working electrode assembly and the biological interface may be increased using a suction cup.

In another embodiment, the adhesive surface of the at least one of the working electrode assembly and a non-working electrode assembly may protrude into an inside of the suction cup, such that, during engagement with the biological interface, the adhesive surface may be pressed further than a tip portion of the suction cup in the direction of the biological interface. In yet another embodiment, the suction cup may include a suction hole between an inside and an outside of the suction cup and a non-return valve coupled to the suction hole for preventing air from flowing from the outside into the inside of the suction cup and for allowing air to flow from the inside to the outside of the suction cup. In this embodiment, air present between the working electrode assembly and/or the non-working electrode assembly and the biological interface may be removed using an external vacuum.

In yet another embodiment, a seal may be provided between the at least one of the working electrode assembly and the non-working electrode assembly and the biological interface.

Thus, improved adhesion may be secured over a longer time period.

In yet another embodiment, the suction cup may be formed integrally with a case of the at least one of the working electrode assembly and the non-working electrode assembly. This arrangement may reduce production costs.

### BRIEF DESCRIPTION OF THE SEVERAL VIEWS OF THE DRAWINGS

In the drawings, identical reference numbers identify similar elements. The sizes and relative positions of elements in the drawings are not necessarily drawn to scale. For example, the shapes of various elements and angles are not drawn to scale, and some of these elements have been arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 is a cross-sectional view of electrode assemblies of an iontophoresis device, according to one illustrated embodiment.

FIGS. 2A, 2B and 2C show a method of mounting a working electrode assembly of FIG. 1, according to one illustrated embodiment.

FIG. 3 is a cross-sectional view of a suction cup of a working electrode assembly provided with a suction hole and a non-return valve, according to one illustrated embodiment.

FIG. 4 shows an iontophoresis device mounted using a seal, according to another illustrated embodiment.

FIG. 5 is a schematic view of one conventional iontophoresis device.

FIG. 6 shows an electrode assembly of the iontophoresis device of FIG. 5.

FIG. 7 is a perspective view of another exemplary iontophoresis device.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures and methods associated with iontophoresis devices, electric power sources, and drug delivery have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

According to the embodiments described herein, the degree of adhesion between the adhesive surface/surfaces of a working electrode assembly and/or a non-working electrode assembly of an iontophoresis device and a biological interface may be increased using a negative pressure device, and the transport efficiency of a drug (e.g., ionic drug) may be similarly increased.

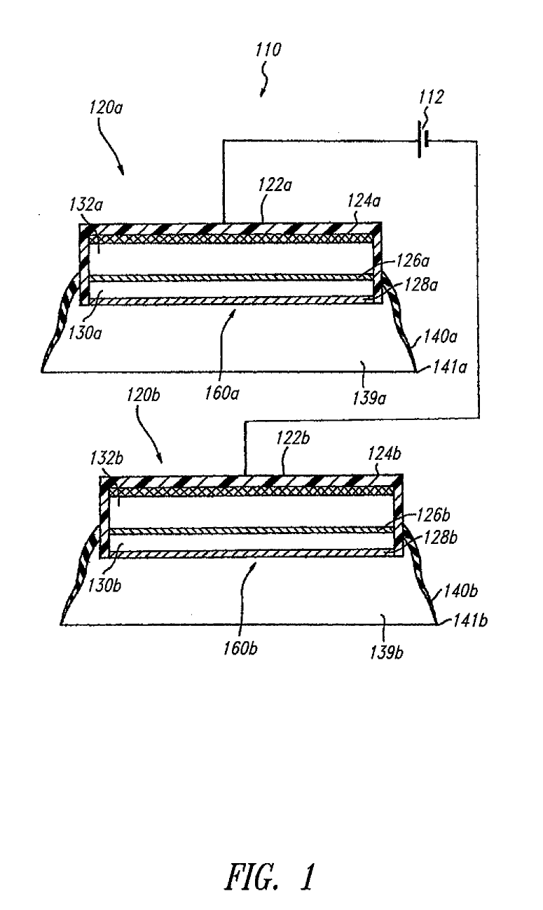

FIG. 1 shows an iontophoresis device 110 including a working electrode assembly 120a and a non-working electrode assembly 120b according to one illustrated embodiment. The working electrode assembly 120a will be described in greater detail below.

For convenience of description, the iontophoresis device 110 will be discussed in the context of administering a drug whose drug component dissociates to cations. Other drugs may be administered in other embodiments. For example, an iontophoresis device for administering a drug whose drug component dissociates to anions may be implemented by switching the polarity of each of: a voltage applied to each electrode; and an ion exchange membrane and an exchange group introduced into an ion exchange resin.

The working electrode assembly 120a may include a backing layer/housing 124a housing: an electrode 122a connected to an electric power source 112 (e.g., a DC electric power source), a buffer solution holding portion 132a adjacent a front surface of the electrode 122a, an anion exchange membrane 126a adjacent a front surface of the buffer solution holding portion 132a, a drug holding portion 130a adjacent a front surface of the anion exchange membrane 126a, and a cation exchange membrane 128a adjacent a front surface of the drug holding portion 130a. In the illustrated embodiment, the backing layer 124a doubles as a case for the working electrode assembly 120a.

Any conductive material may be used for the electrode 122a. When a buffer solution holding portion 132a is present, as in the illustrated embodiment, a carbon electrode may be used.

The buffer solution holding portion 132a may hold a buffer solution (e.g., an electrolyte solution) for maintaining energization of the iontophoresis device 110 over a longer time period. A phosphate buffered saline may, for example, serve as the buffer solution. An oxidation reaction may take place since the working electrode assembly 120a is connected to an anode of the electric power source 112. In certain embodiments, an electrolyte that is more easily oxidized than water may therefore be used, such as: an inorganic compound (e.g., ferrous phosphate or ferric phosphate); ascorbic acid (vitamin C) or sodium ascorbate; an organic acid (e.g., lactic acid, oxalic acid, malic acid, succinic acid, or fumaric acid) and/or a salt thereof; or a mixture of the above. By using such an electrolyte, the generation of gas due to the electrolysis of water, and a corresponding increase in conductive resistance or fluctuation in pH value may be mitigated or prevented. Of course, other buffer solutions may be used in other embodiments.

The anion exchange membrane 126a may include an ion exchange membrane for permitting passage of anions. For example, a NEOSEPTA (AM-1, AM-3, AMX, AHA, ACH, or ACS) manufactured by Tokuyama Co., Ltd may be used as the anion exchange membrane 126a. In another embodiment, the anion exchange membrane 126a may include a semipermeable film comprising a polyolefin resin, a vinyl chloride-based resin, a fluorine-based resin, a polyamide resin, or a polyimide resin, the film having cavities of which a whole or a part are polymerized with an anion exchange resin. In such an embodiment, the cavities may be filled with the anion exchange resin by: impregnating the cavities of the semi-permeable film with a solution prepared by blending a crosslinkable monomer such as styrene-divinylbenzene or chloromethylstyrene-divinylbenzene with a polymerization initiator; polymerizing the resultant; and introducing into the polymer an anion exchange group, such as a primary amino group, a secondary amino group, a tertiary amino group, a quaternary ammonium group, a pyridyl group, an imidazole group, a quaternary pyridinium group, or a quaternary imidazolium group.

In the illustrated embodiment, the drug holding portion 130a may hold a solution of a drug whose drug component dissociates to cations as a result of dissolution. In another embodiment, in which the working electrode assembly is connected to a cathode of an electric power source, the drug holding portion may hold a solution of a drug whose drug component dissociates to anions.

The cation exchange membrane 128a may include an ion exchange membrane for permitting passage of cations. For example, the cation exchange membrane 128a may be a NEOSEPTA (CM-1, CM-2, CMX, CMS, or CMB) manufactured by Tokuyama Co., Ltd. In another embodiment, the cation exchange membrane 128a may include a semi-permeable film composed of a polyolefin resin, a vinyl chloride-based resin, a fluorine-based resin, a polyamide resin, or a polyimide resin, the film having cavities of which a whole or a part are filled with a cation exchange resin. In such an embodiment, the cavities may be filled with the cation exchange resin by: impregnating the cavities of the semi-permeable film with a solution prepared by blending a crosslinkable monomer such as styrene-divinylbenzene or chloromethylstyrene-divinylbenzene with a polymerization initiator; polymerizing the resultant; and introducing into the polymer a cation exchange group, such as a sulfonic group, a carboxylic group, or a phosphonic group.

The backing layer 124a may function as a primary structural element of the working electrode assembly 120a. In addition, the backing layer 124a may serve as a partition wall from the outside, that is, as a protective cover.

In one embodiment, the working electrode assembly 120a includes a negative pressure device, such as a suction cup 140a. In one embodiment, the suction cup 140a at least partially surrounds the adhesive surface 160a of the working electrode assembly 120a. In FIG. 1, the suction cup 140a is arranged on a side surface of the backing layer 124a. However, the arrangement is not thus limited. For example, the suction cup 140a may also be formed integrally with the backing layer 124a.

In one embodiment, the suction cup 140a is integral with the working electrode assembly 120a and comprises a material having an elastic restoring force, such as silicon gum.

As illustrated, the adhesive surface 160a of the working electrode assembly 120a may project into an inside region defined by the suction cup 140a. That is, the adhesive surface 160a may be formed in such a manner that it can be pressed into a biological interface more deeply than a tip portion 141a of the suction cup 140a with external pressure.

In one embodiment, the working electrode assembly 120a has one anion exchange membrane and one cation exchange membrane. However, other configurations are also possible. For example, only the cation exchange membrane may be used, or a bipolar membrane may be used. In addition, the buffer solution holding portion 132a is included in the illustrated embodiment, but may be omitted in other embodiments.

Below, the action of the iontophoresis device 110 will be described.

When a current from the electric power source 112 propagates to the electrode 122a of the working electrode assembly 120a, the buffer solution in the buffer solution holding portion 132a may be oxidized. As a result, a balance between cations and anions in the buffer solution holding portion 132a may be lost (e.g., the number of cations may increase). Cations of the buffer solution holding portion 132a may then start to move toward the drug holding portion 130a in order to correct the imbalance. Due to the charge of the buffer solution, anions of the drug holding portion 130a may also start to move toward the buffer solution holding portion 132a. However, owing to the presence of the anion exchange membrane 126a between the buffer solution holding portion 132a and the drug holding portion 130a, cations may be unable to pass or may pass only in relatively small quantities, while anions are permitted passage. Thus, relatively little movement of cations from the buffer solution holding portion 132a to the drug holding portion 130a is permitted, while the movement of anions from the drug holding portion 130a to the buffer solution holding portion 132a is much greater. As a result, a balance between cations and anions in the drug holding portion 130a may be lost. Cations of the drug holding portion 130a may therefore start to move toward the biological interface through the cation exchange membrane 128a in order to correct this imbalance. Cations may then be selectively passed by the cation exchange membrane 128a, and move to the side of the biological interface.

If the adhesive surface 160a of the working electrode assembly 120a, including the cation exchange membrane 128a, sufficiently adheres to a biological interface (such as a skin or a mucosa), an increase in contact area may facilitate the movement of cations (e.g., drug ions) permitted passage across the cation exchange membrane 128a to the side of the biological interface. That is, the transport efficiency of drug ions may increase.

In one embodiment, the degree of adhesion between the adhesive surface 160a, including the cation exchange membrane 128a, and the biological interface may increase owing to the presence of the suction cup 140a.

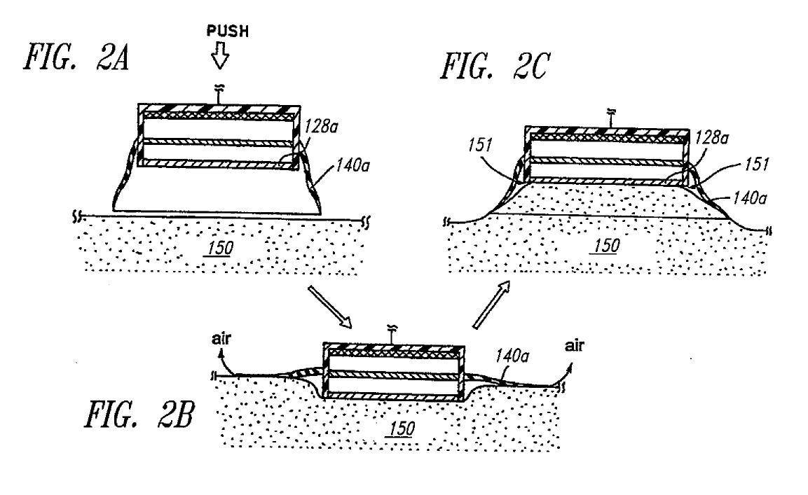

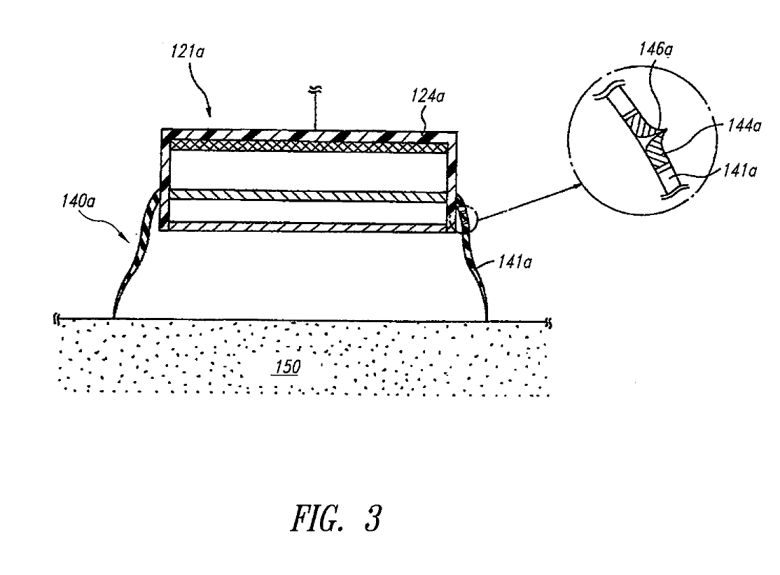

FIGS. 2A, 2B and 2C illustrate one exemplary method of mounting the working electrode assembly 128a.

At first, the working electrode assembly 120a having the suction cup 140a may be placed against a site on skin 150 or on another biological interface. In this state, as shown in FIG. 2A, the working electrode assembly 120a may then be lightly pressed from one side of the working electrode assembly 120a (a side opposite the skin 150) against the side of the skin 150. Thus, as shown in FIG. 2B, the adhesive surface 160a of the working electrode assembly 120a may be pressed further in the direction of the skin 150 than the tip portion 141a of the suction cup 140a. At the same time, air between the adhesive surface 160a of the working electrode assembly 120a and the skin 150 (that is, air present inside the suction cup 140a) may be exhausted to the outside. Once the external pressure applied to the working electrode assembly 120a is removed, a negative pressure relative to the atmosphere may be maintained between the working electrode assembly 120a and the skin 150 by the negative pressure device. As a result, the skin 150 may remain in close contact with a front side of the adhesive surface 160a and, therefore, to a front side of the cation exchange membrane 128a of the working electrode assembly 120a (illustrated in FIG. 2C). The negative pressure may improve an adhesion between the adhesive surface 160a of the working electrode assembly 120a and the biological interface.

In some embodiments, the adhesive surface 160a of the working electrode assembly 120a may be formed so as to project into the inside of the suction cup 140a, so that the adhesive surface 160a and the skin 150 may contact each other when a negative pressure is successfully generated.

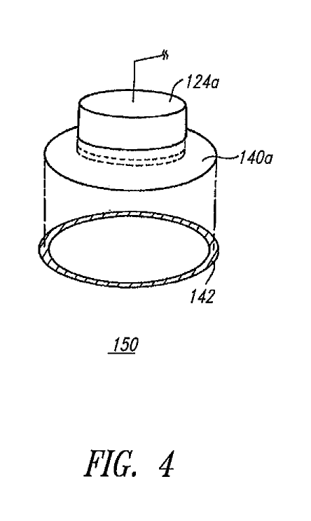

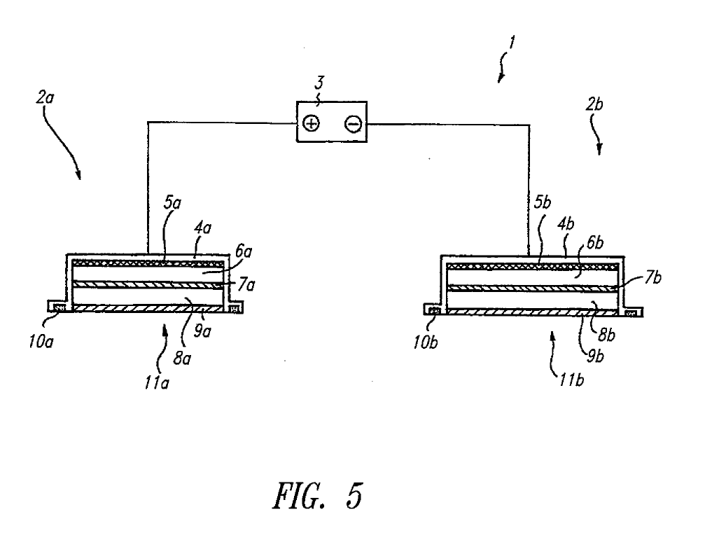

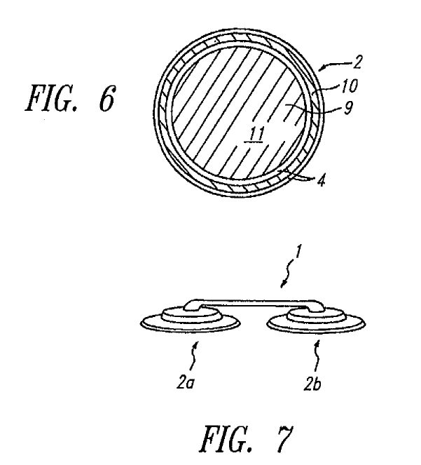

In one embodiment, negative pressure may be generated and maintained using the device shown in FIG. 3, instead of by a suction cup alone.

Part of the suction cup 140a of the working electrode assembly 120a shown in FIG. 3 may form a suction hole 146a for establishing communication between an inside and an outside of the suction cup 140a. The suction hole 146a may comprise a non-return valve 144a so that air removed by an external vacuum may not return. In one embodiment, adhesion between the adhesive surface 160a of the working electrode assembly 120a and the skin 150 may be increased by removing air from the suction cup 140a through the suction hole 146a.

In another embodiment, when one desires to mount the iontophoresis device 110, a negative pressure generated by one of the above-described negative pressure devices may not be maintained well owing to hairs present on a certain site of a biological interface or owing to the dryness of a surface such as skin. Therefore, a seal 142 of the suction cup 140a having a smooth surface may be used to mitigate and/or prevent air leaks, as shown in FIG. 4. The seal 142 may be stuck in advance to the side of the biological interface where a user desires to mount the iontophoresis device 110, and the working electrode assembly 120a of the iontophoresis device 110 may be mounted in alignment with the seal 142. As a result, a higher degree of adhesion may be maintained over a long time period.

Although not shown, an inner surface of the suction cup 140a may also be provided with an adhesive portion capable of adhering to a biological interface, such as skin. Thus, adhesion between the adhesive surface 160a of the working electrode assembly 120a and the biological interface may be increased by a synergistic effect between the adhesive strength of the inner surface of the suction cup 140a and the negative pressure generated by the suction cup 140a.

In the foregoing description, attention has been paid particularly to the working electrode assembly. Of course, the description is similarly applicable to the non-working electrode assembly. In such case, the transport efficiency of a drug ion may be additionally increased.

The above embodiments are applicable not only to an iontophoresis device for a human body but also an iontophoresis device to be used on a wide variety of animals and plants.

### DESCRIPTION OF REFERENCE NUMERALS

110 IONTOPHORESIS DEVICE
112 ELECTRIC POWER SOURCE
120a WORKING ELECTRODE ASSEMBLY
122a ELECTRODE
124a BACKING LAYER
126a ANION EXCHANGE MEMBRANE
128a CATION EXCHANGE MEMBRANE
130a DRUG HOLDING PORTION
132a BUFFER SOLUTION HOLDING PORTION
139a NEGATIVE PRESSURE DEVICE
140a SUCTION CUP
142 SEAL
144a NON-RETURN VALVE
146A SUCTION HOLE
150 SKIN (BIOLOGICAL INTERFACE)
160a ADHESIVE SURFACE

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

The various embodiments described above can be combined to provide further embodiments. From the foregoing it will be appreciated that, although specific embodiments have been described herein for purposes of illustration, various modifications may be made without deviating from the spirit and scope of the teachings. Accordingly, the claims are not limited by the disclosed embodiments.

We claim:

1. An iontophoresis devices comprising:
a working electrode assembly and a non-working electrode assembly for administering a drug by iontophoresis, each of the working electrode assembly and the non-working electrode assembly including an adhesive surface configured to adhere to a biological interface;
a DC electric power source connected to the working electrode assembly and the non-working electrode assembly; and
a negative pressure generating device integral with at least one of the working electrode assembly and the non-working electrode assembly and configured to maintain a negative pressure between the at least one of the working electrode assembly and the non-working electrode assembly and the biological interface.

2. The iontophoresis device of claim 1, wherein the negative pressure device comprises a suction cup at least partially surrounding the adhesive surface of the at least one of the working electrode assembly and the non-working electrode assembly; and
wherein the suction cup is configured to generate and maintain the negative pressure when the suction cup returns to an original shape due to an elastic restoring force of the suction cup after the suction cup is pressed in toward the biological interface.

3. The iontophoresis device of claim 1, wherein the negative pressure device comprises a suction cup at least partially surrounding the adhesive surface of the at least one of the working electrode assembly and the non-working electrode assembly, and wherein the adhesive surface protrudes into an inside of the suction cup, such that, during engagement with the biological interface, the adhesive surface can be pressed further than a tip portion of the suction cup in a direction of the biological interface.

4. The iontophoresis device of claim 1, wherein the negative pressure device comprises a suction cup at least partially surrounding the adhesion surface of the working electrode assembly and the non-working electrode assembly, and the suction cup includes a suction hole between an inside and an outside of the suction cup and a non-return valve coupled to the suction cup for preventing air from flowing from the outside into the inside of the suction cup and for allowing air to flow from the inside to the outside of the suction cup.

5. The iontophoresis device of claim 1, wherein a seal is provided between the at least one of the working electrode assembly and the non-working electrode assembly and the biological interface.

6. The iontophoresis device of claim 1, wherein the negative pressure generating device is integral with a case of the at least one of the working electrode assembly and the non-working electrode assembly.

7. The iontophoresis device of claim 2, wherein the suction cup includes adhesive along an inside surface.
